(19) United States
(12) Patent Application Publication
(10) Pub. No.: US 2018/0037860 A1
(43) Pub. Date: Feb. 8, 2018

(54) LIQUID TRANSFERRING APPARATUS AND LIQUID TRANSFERRING METHOD

(71) Applicant: Panasonic Corporation, Osaka (JP)

(72) Inventors: HISATO NAKAJIMA, Osaka (JP); TAKESHI ANDO, Kyoto (JP); NORIHIRO SHIBATA, Osaka (JP)

(21) Appl. No.: 15/605,953

(22) Filed: May 26, 2017

(30) Foreign Application Priority Data
Aug. 5, 2016 (JP) 2016-155014

(51) Int. Cl. C12M 1/26; B01L 3/02

(52) U.S. Cl. CPC C12M 33/04; B01L 3/0227; B01L 2400/0478; B01L 2300/0832

(57) ABSTRACT

A liquid transferring apparatus sucks up or discharges liquid by moving piston of syringe vertically including first motor which actuates piston by supporting flange pulling-up portion to be vertically movable and by pulling up flange portion of piston from below via flange pulling-up portion; second motor which supports flange pushing portion to be vertically movable and actuates piston so as to push flange portion of piston from above via flange pushing portion; cylinder fixing portion which fixes cylinder of syringe; and mechanical frame.

### TECHNICAL FIELD

The technical field relates to a liquid transferring apparatus and a liquid transferring method.

### BACKGROUND

A related art liquid transferring apparatus used in cell culture includes a piston actuated by a motor to move a flange of a syringe for sucking/injecting a liquid from a gripped container (see Japanese Patent Examined Publication No. 6-34825).

FIG. 11 is a view illustrating a liquid transferring apparatus of the related art disclosed in Japanese Patent Examined Publication No. 6-34825.

In FIG. 11, cylinder 3 of syringe 2 is set on lower support member 11 provided on base 1, and cylinder 3 of syringe 2 is fixed by upper support member 12, while flange portion 5a of piston 5 is set on pushing member 6.

Pushing member 6 is fixed to movable block 17, and advances/retracts together with movable block 17 by movable block 17 advancing/retracting by actuation of motor 16. Accordingly, since pushing member 6 is actuated so as to push/pull up set piston 5 to/from cylinder 3, suction/injection of liquid is realized.

### SUMMARY

However, in the configuration of the related art, since flange portion 5a of piston 5 can be attached to/removed from pushing member 6, a gap is always generated between flange portion 5a and a portion sandwiching flange portion 5a. As a result, there is a problem that the gap affects the accuracy of the amount of liquid to be sucked/injected.

For example, in a case where a syringe sucks up 3 ml of liquid when piston 5 is pulled up by 50 mm from a pushed in state and the syringe injects liquid after sucking up liquid, if a gap of 0.1 mm is generated between (between 5a and 6a) flange portion 5a of piston 5 and a portion of pushing member 6 in which flange portion 5a is sandwiched, by calculation, an error of 3 ml÷50 mm×0.1 mm=0.006 ml (6 μl) is generated in the amount of liquid to be injected.

In view of the above problem, as well as other concerns, a liquid transferring apparatus and a liquid transferring method which is preferably used in a cell culture apparatus, eliminates an error caused by a gap existing between a flange portion of a piston and a portion sandwiching the flange portion of the related art, and liquid can be sucked up/injected with high liquid quantity accuracy.

In order to achieve the object described above, according to main aspect of the disclosure, in a cell culture apparatus which sucks up or discharges a liquid by moving a piston of a syringe vertically, there is provided a liquid transferring apparatus including a first motor which actuates the piston by supporting a flange pulling-up portion to be vertically movable and by pulling up a flange portion of the piston from below via the flange pulling-up portion; a second motor which supports a flange pushing portion to be vertically movable and actuates the piston so as to push the flange portion of the piston from above via the flange pushing portion; a cylinder fixing portion which fixes a cylinder of the syringe; and a mechanical frame which supports the first motor, the second motor, and the cylinder fixing portion.

In the liquid transferring apparatus, when liquid is sucked up, the second motor is actuated in a direction in which liquid is sucked up by a stroke amount corresponding to an amount of liquid to be sucked up and then the first motor is also actuated in a direction in which liquid is sucked up until the flange pulling-up portion is hit against the flange portion and is stopped. Further, when liquid is injected, the first motor is actuated in a direction in which liquid is injected by a stroke amount corresponding to an amount of liquid to be injected and then the second motor is also actuated in a direction in which liquid is injected until the flange pushing portion is hit against the flange portion and is stopped.

As described above, according to the liquid transferring apparatus provided in the cell culture apparatus of the disclosure, since gaps between the flange portion and the flange pulling-up portion and between the flange portion and the flange pushing portion can be eliminated at the time of liquid suction/injection, suction/injection of liquid can be performed with high accuracy.

### BRIEF DESCRIPTION OF THE DRAWINGS

FIG. 1A is a front view illustrating a liquid transferring apparatus according to an embodiment of the disclosure.

FIG. 1B. is a side view illustrating the liquid transferring apparatus according to the embodiment of the disclosure.

FIG. 2 is a view illustrating a positional relationship between a syringe and a motor.

FIG. 3A is a view illustrating a state where a claw of a cylinder fixing portion is opened.

FIG. 3B is a view illustrating a state where a cylinder is fixed by the claw of the cylinder fixing portion being closed.

FIG. 4 is a view illustrating a state where the piston is pushed into the cylinder.

FIG. 5 is a view illustrating a state in which the piston is pushed into the cylinder to a tip thereof and the flange portion is sandwiched between the flange pulling-up portion and the flange pushing portion without any gap.

FIG. 6 is a view illustrating a state where the second motor is actuated to a side opposite to the tip of the cylinder by a stroke amount corresponding to an amount of liquid to be sucked up.

FIG. 7 is a view illustrating a state in which the flange pulling-up portion is in contact with the flange portion, the flange pushing portion and the flange portion are sandwiched between each other without any gap, and a first motor is stopped.

FIG. 8 is a view illustrating a state where the first motor is actuated to a tip side of the cylinder by a stroke amount corresponding to an amount of liquid to be injected.

FIG. 9 is a view illustrating a state in which the flange pushing portion is in contact with the flange portion, the flange pulling-up portion and the flange portion are sandwiched with each other without any gap, and the second motor is stopped.

FIG. 10 is a view illustrating a state in which all the liquid in the cylinder is discharged.

FIG. 11 is a view illustrating a structure of a liquid suction/injection mechanism of the related art.

### DESCRIPTION OF EMBODIMENTS

Hereinafter, embodiment of the disclosure will be described with reference to the drawings.

### Embodiment

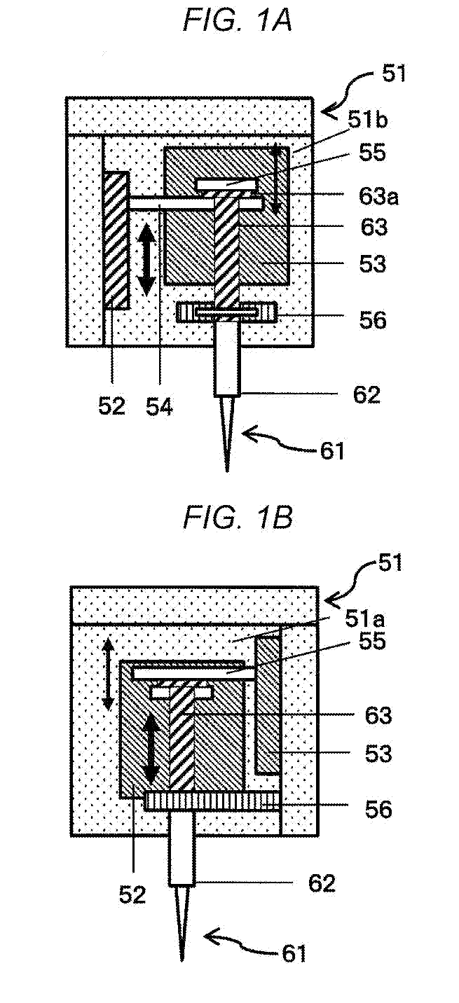

FIG. 1A is a front view illustrating a liquid transferring apparatus which is provided in a cell culture apparatus according to an embodiment of the disclosure, and FIG. 1B is a side view thereof.

The cell culture apparatus includes first motor 52, second motor 53, syringe 61, flange pulling-up portion 54, flange pushing portion 55, cylinder fixing portion 56, and mechanical frame 51, as a liquid transferring apparatus.

Mechanical frame 51 is a base member which supports and fixes syringe 61 (cylinder fixing portion 56), first motor 52, and second motor 53.

Syringe 61 includes cylinder 62 and piston 63 which is actuated in cylinder 62. Further, flange portion 63a is provided at an upper end of piston 63 of syringe 61. Piston. 63 is vertically actuated by power being transferred to flange portion 63a via flange pulling-up portion 54 or flange pushing portion 55.

First motor 52 supports flange pulling-up portion 54 to be vertically movable and actuates piston 63 so as to pull up flange portion 63a of piston 63 from below via flange pulling-up portion 54.

Second motor 53 supports flange pushing portion 55 to be vertically movable and actuates piston 63 so as to push flange portion 63a of piston 63 from above via flange pushing portion 35.

In addition, first motor 52 and second motor 53 are, for example, linear motors, which are driven by a driving power supplied by a control device (not illustrated) provided outside.

Here, flange pulling-up portion 54 includes a flat surface on an upper surface thereof. When flange pulling-up portion 54 is moved by first motor 52, the upper surface of flange pulling-up portion 54 abuts against a lower surface of flange portion 63a of piston 63 so as to be in close contact therewith.

Further, flange pushing portion 55 includes a flat surface on the lower surface. When flange pushing portion 55 is moved by second motor 53, the lower surface of flange pushing portion 55 abuts against an upper surface of flange portion 63a of piston 63 so as to be in close contact therewith.

Mechanical frame 51 has, for example, an L-shaped external shape. For example, mechanical frame 51 is disposed so that first surface 51a and second surface 51b between which an intersect ion line extends along the vertical direction and which are orthogonal to each other are formed.

In FIG. 1A, first motor 52 is attached to first surfaces 51a of one side of mechanical frame 51 and second motor 53 is attached to second surface 51b of one side of mechanical frame 51 which forms an angle of 90 degrees with first surface 51a.

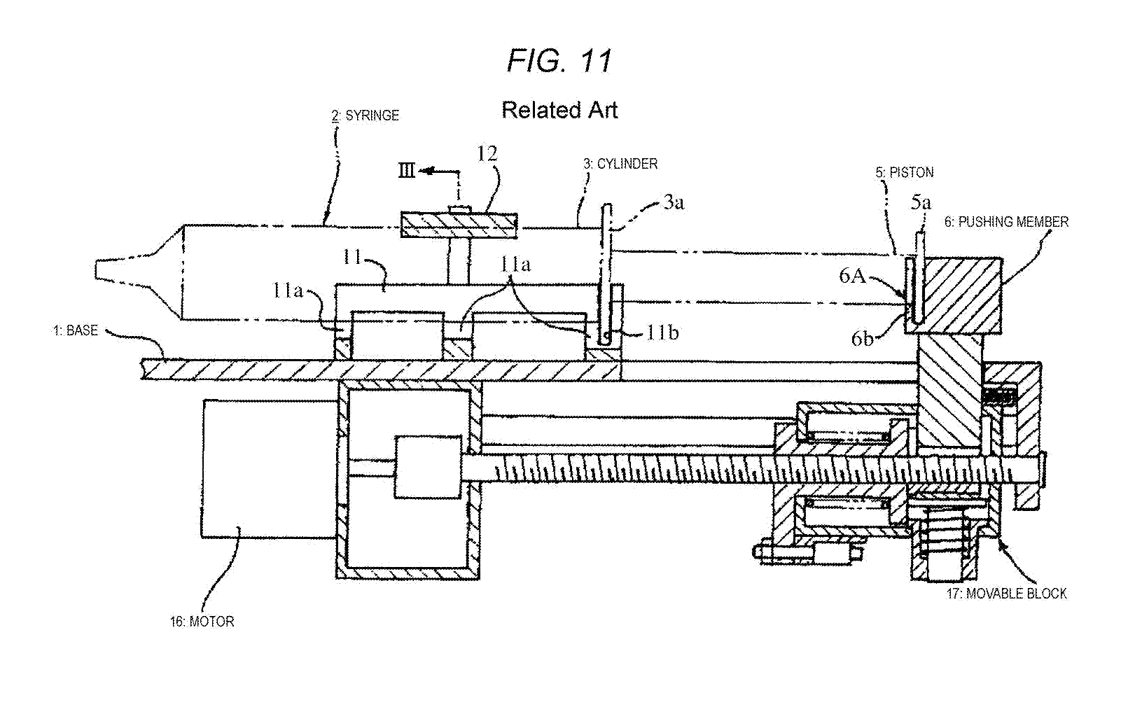

Although first motor 52 and second motor 53 can also be configured by motors which change a rotational motion into a linear motion using the screw shaft as shown in FIG. 11 which explains an example of related art, a linear motor using a permanent magnet and an electromagnetic coil is preferable because the linear motor can be made thinner and a main body of the liquid transferring apparatus can be miniaturized.

In addition, syringe 61 is attached to mechanical frame 51 via cylinder fixing portion 56 which fixes cylinder 62. In the present embodiment, cylinder fixing portion 56 is attached to same second surface 51b as second motor 53. In addition, cylinder fixing portion 56 can be attached to same first surface 51a as first motor 52.

With the liquid transferring apparatus, syringe 61 is mounted, liquid is sucked up/injected, the residual liquid is discharged, and syringe 61 is removed in the following order.

First, syringe 61 is mounted in the following order.

When syringe 61 is mounted on mechanical frame 51, the upper and lower surfaces of flange portion 63a are disposed between the lower surface of flange pushing portion 55 and the upper surface of flange pulling-up portion 54. At this time, first, first motor 52 and second motor 53 are actuated so that the gap between the upper surface of flange pulling-up portion 54 and the lower surface of flange pushing portion 55 is equal to or more than the width of flange portion 63a.

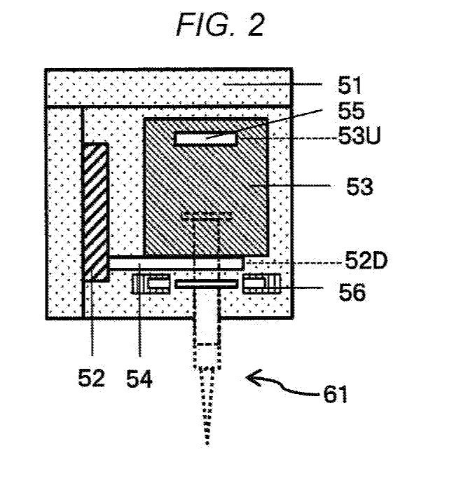

More preferably, as illustrated in FIG. 2, in order to facilitate mounting of syringe 61, flange pulling-up portion 54 is moved to a position of lower limit 52D and first motor 52 and second motor 53 are actuated in order to move flange pushing portion 55 to a position of upper limit 53U.

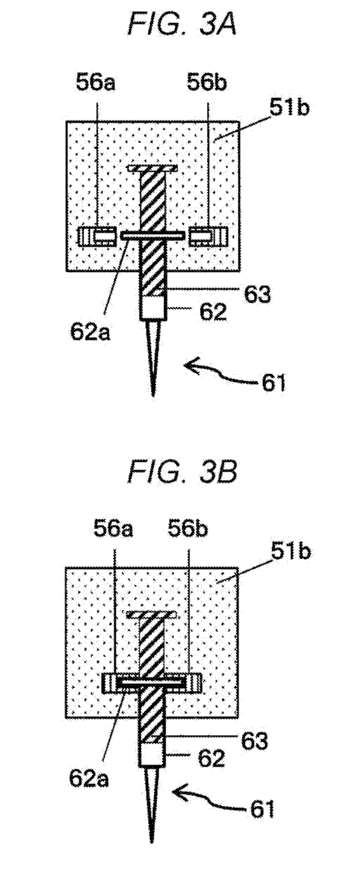

After syringe 61 is positioned with respect to mechanical frame 51, cylinder 62 is fixed by cylinder fixing portion 56 (FIG. 3A and FIG. 3B). Generally, cylinder 62 has collar 62a having a circular width at an end opposite to the tip thereof. In the embodiment, collar 62a is inserted and sandwiched in the groove of claws 56a and 56b by collar 62a of cylinder 62 being closed by claws 56a and 56b provided with grooves in the laterally direction. Accordingly, cylinder 62 is fixed to cylinder fixing portion 56 and thus syringe 61 is mounted.

The suction of the liquid fay syringe 61 is performed in the following order.

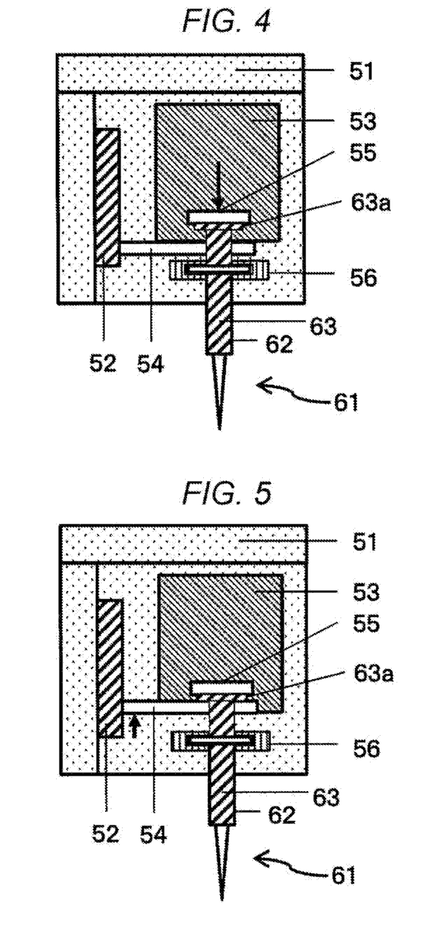

First, second motor 53 is actuated to move flange pushing portion 55 toward the tip side of cylinder 62 . At this time, the lower surface of flange pushing portion 55 is in contact with the upper surface of flange portion 63a and pushes flange portion 63a, and thus piston 63 is stopped in a state of being pushed into cylinder 62 to the tip thereof (FIG. 4).

Next, first motor 52 is actuated to move flange pulling-up portion 54 toward a side opposite to the tip of cylinder 62 (FIG. 5). At this time, the upper surface of flange pulling-up portion 54 is in contact with the lower surface of flange portion 63a and pulls up flange portion 63a and thus piston 63 is pulled up.

However, in FIG. 5, when flange pulling-up portion 54 is moved toward a side opposite to the tip of cylinder 62 by first motor 52, torque is applied from second motor 53 to flange pushing portion 55. For example, when flange pulling-up portion 54 is moved, static torque is applied from second motor 53 to flange pushing portion 55. At this time, the torque amount [N·m] applied to flange pulling-up portion 54 by first motor 52 is set to be less than the torque amount [N·m] applied to flange pushing portion 55 by second motor 53. Accordingly, the piston 63 is pushed into cylinder 62 to the tip thereof and flange portion 63a is sandwiched between flange pulling-up portion 54 and flange pushing portion 55 without any gap.

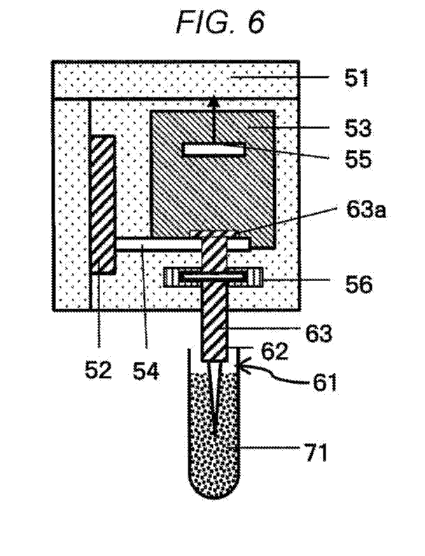

In this state, the tip of cylinder 62 is immersed into liquid 71 to be sucked up and first, flange pushing portion 55 is moved to a side opposite to the tip of cylinder 62 by the stroke amount corresponding to the amount of liquid to be sucked, by second motor 53 (FIG. 6).

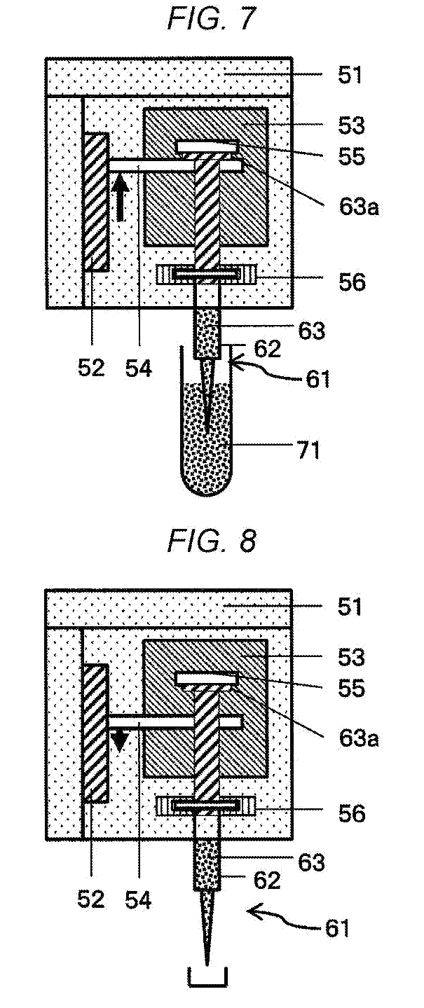

Thereafter, by actuation of first motor 52, flange pulling-up portion 54 is moved to a side opposite to the tip of cylinder 62 (FIG. 7). Flange pulling-up portion 54 is moved until being pushed by flange portion 63a and stopped. By the actuation of first motor 52, flange pulling-up portion 54 pulls up flange portion 63a and piston 63 and the liquid is sucked up into cylinder 62.

In FIG. 7, however, when flange pulling-up portion 54 is moved to a side opposite to the tip of cylinder 62 by first motor 52, torque is applied from second motor 53 to flange pushing portion 55. For example, when flange pulling-up portion 54 is moved, static torque is applied from second motor 53 to flange pushing portion 55. At this time, the torque amount [N·m] applied to flange pulling-up portion 54 by first motor 52 is desirably set to be less than the torque amount [N·m] applied to flange pushing portion 55 by second motor 53. According to this, flange pulling-up portion 54 is stopped in a state of being in contact with flange portion 63a pushed by the flange pushing portion 55.

Accordingly, flange portion 63a is sandwiched between flange pulling-up portion 54 and flange pushing portion 55 without any gap. Thus, the error of the liquid amount due to the gap which is the problem of the example of the related art can be eliminated, and thus liquid can be sucked up with high accuracy.

The injection of the liquid by syringe 61 is performed in the following order.

In a case where the sucked liquid is injected, first motor 52 is actuated to move flange pulling-up portion 54 to the tip side of cylinder 62 by the stroke amount corresponding to the amount of liquid to be injected (FIG. 8).

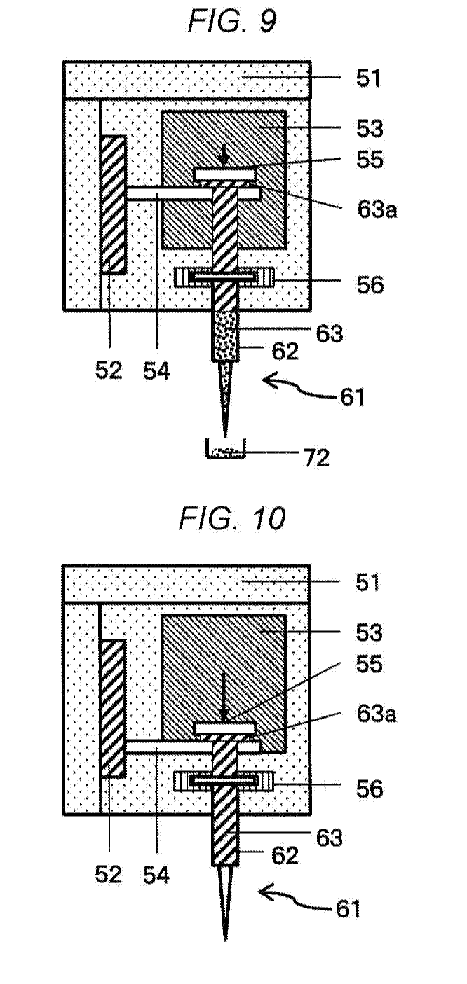

Thereafter, by the actuation of second motor 53, flange pushing portion 55 is moved to the tip side of cylinder 62 (FIG. 9). Flange pushing portion 55 is moved until being pushed by flange portion 63a and stopped. By the actuation of second motor 53, flange pushing portion 55 pushes down flange portion 63a and piston 63 and the liquid is injected.

However, in FIG. 9, when flange pushing portion 55 is moved to the tip side of cylinder 62 by second motor 53, torque is applied from first motor 52 to flange pulling-up portion 54. For example, when flange pushing portion 55 is moved, static torque is applied from first motor 52 to flange pulling-up portion. 54. At this time, the torque amount [N·m] which is applied to flange pushing portion 55 by second motor 53 is desirably set to be less than the torque amount [N·m] which is applied to flange pulling-up portion 54 by first motor 52. According to this, flange pushing portion 55 is stopped in a state of being in contact with flange portion 63a supported by flange pulling-up portion 54.

Accordingly, flange portion 63a is sandwiched between flange pulling-up portion 54 and flange pushing portion 55 without any gap. Thus, the error of the liquid amount due to the gap which is the problem of the example of the related art can be eliminated, and thus liquid can be sucked up with high accuracy.

After the desired injection is completed, the discharge of the residual liquid remaining in cylinder 62 is performed in the following order.

When all the liquid in cylinder 62 is discharged, excitation of first motor 52 is cut off and second motor 53 is actuated by torque control toward the tip of cylinder 62. In a state where piston 63 is pushed into cylinder 62 to the tip thereof, although second motor 53 is stopped, it is possible to set a state where all of the liquid in cylinder 62 is discharged (FIG. 10).

Finally, syringe 61 is removed in the following order.

In a case where syringe 61 is removed, in the positions of first motor 52 and second motor 53, when the gap between flange pulling-up portion 54 and flange pushing portion 55 is equal to or more than the width of flange portion 63a, although syringe 61 can be removed, in order to facilitate the removal, it is preferable to position flange pulling-up portion 54 at lower limit 52D and flange pushing portion 55 at upper limit 53U. This is the same as when being mounted.

Simultaneously, the fixing of cylinder 62 fixed by cylinder fixing portion 56 is released. By opening claws 56a and 56b which sandwich collar 62a of cylinder 62, the fixing of the cylinder is released and becomes a state of being removable.

The cell culture apparatus including the liquid transferring apparatus described herein can be used in a process which is referred to as medium exchange.

Although cells are cultured in a culture container such as a dish or a well plate filled with a medium which is culture liquid, the medium needs to be replaced within a certain period of time, and the medium exchange is a process which sucks up the old medium in the culture container, discards the old medium and injects fresh medium.

In the cell culture apparatus provided with the liquid transferring apparatus of the disclosure, medium exchange can be performed in the following order.

First, syringe 61 is mounted on the liquid transferring apparatus, after being mounted, the liquid transferring apparatus is moved so that the tip of syringe 61 is immersed into the medium of the culture container, and the medium is sucked up. For the dish and the well plate, since recommended amount of injection liquid is defined and the amount of liquid which is injected is determined in advance, the recommended amount is referred to as an amount of liquid to be sucked up.

After the sucking up is completed, the liquid transferring apparatus is moved from the culture container, piston 63 is pushed into cylinder 62, and the sucked up medium is discarded in a state of being all discharged. Thereafter, syringe 61 is removed from the liquid transferring apparatus.

Next, new syringe 61 is mounted on the liquid transferring apparatus. The purpose of replacing syringe 61 is to prevent contamination.

After new syringe 61 is mounted, the liquid, transferring apparatus is moved so that the tip of syringe 61 is immersed into fresh medium and the medium is sucked up. As described above, for the dish and the well plate, since the recommended amount of injection liquid is defined and the amount of liquid to be injected is determined, the amount of liquid to be injected is referred to as an amount of liquid to be sucked up.

After suction of the fresh medium, the liquid transferring apparatus is moved so that a fresh medium is injected into the culture container and injects the amount of liquid described above.

The liquid transferring apparatus is moved from the culture container, piston 63 is pushed in cylinder 62, all the remaining medium is discharged and thus the remaining medium is discarded and thereafter syringe 61 is removed from the liquid transferring apparatus.

As described above, although specific example of the disclosure is described in detail, the example is merely an example and does not limit the scope of the claims. Techniques described in the claims include those in which the specific example exemplified above is variously modified and changed.

Since the liquid transferring apparatus provided in the cell culture apparatus of the disclosure can eliminate the gap between the flange and the flange pulling-up portion or the flange pushing portion at the time of liquid suction/injection, the error of the liquid amount due to the gap which is the problem of the example of the related art can be eliminated, liquid suction/injection can be performed with high accuracy, and thus the liquid transferring apparatus can be also applied to the application of cell culture process automation.

What is claimed is:

1. A liquid transferring apparatus which sucks up or discharges liquid by moving a piston of a syringe vertically, the apparatus comprising:
a first motor which actuates the piston by supporting a flange pulling-up portion to be vertically movable and by pulling up a flange portion of the piston from below via the flange pulling-up portion;
a second motor which supports a flange pushing portion to be vertically movable and actuates the piston so as to push the flange portion of the piston from above via the flange pushing portion;
a cylinder fixing portion which fixes a cylinder of the syringe; and
a mechanical frame which supports the first motor, the second motor, and the cylinder fixing portion.

2. The liquid transferring apparatus of claim 1,
wherein when the liquid is sucked up by the syringe, the first motor moves the flange pulling-up portion upward until an upper surface of the flange portion of the piston abuts against a lower surface of the flange pushing portion, and a lower surface of the flange portion of the piston abuts against an upper surface of the flange pulling-up portion.

3. The liquid transferring apparatus of claim 2,
wherein when liquid is sucked up by the syringe, the first motor moves the flange pulling-up portion upward by a torque amount which is less than a torque amount which is applied to the flange pushing portion by the second motor.

4. The liquid transferring apparatus of claim 2,
wherein when liquid is sucked up by the syringe, the first motor moves the flange pulling-up portion upward after the second motor moves the flange pushing portion toward a tip of the cylinder until the piston is pushed into the cylinder to the tip thereof, and moves the flange pulling-up portion upward after the second motor moves the flange pushing portion toward a side opposite to the tip of the cylinder by a stroke amount corresponding to an amount of liquid to be sucked up.

5. The liquid transferring apparatus of claim 1,
wherein when liquid is discharged by the syringe, the second motor moves the flange pushing portion downward until the upper surface of the flange portion of the piston abuts against the lower surface of the flange pushing portion, and the lower surface of the flange portion of the piston abuts against the upper surface of the flange pulling-up portion.

6. The liquid transferring apparatus of claim 5,
wherein when liquid is discharged by the syringe, the second motor moves the flange pushing portion downward by a torque amount which is less than a torque amount which is applied to the flange pulling-up portion by the first motor.

7. The liquid transferring apparatus of claim 5,
wherein when liquid is discharged by the syringe, the second motor moves the flange pushing portion downward until the piston is pushed into the cylinder to the tip thereof by the flange pushing portion being moved toward the tip of the cylinder.

8. A liquid transferring method which uses a liquid transferring apparatus including a first motor which actuates a piston by pulling up a flange portion of a syringe from below and a second motor which actuates the piston by pushing the flange portion of the syringe from above, the method comprising:
setting, when medium is sucked up, a torque amount which is applied to the flange portion by the first motor to be less than a torque amount which is applied to the flange portion by the second motor; and
setting, when the medium is injected, a torque amount which is applied to the flange portion by the second motor to be less than a torque amount which is applied to the flange portion by the first motor.
